(19) United States
(12) Patent Application Publication
(10) Pub. No.: US 2013/0206655 A1
(43) Pub. Date: Aug. 15, 2013

(54) Wearable System for Performing Purifying Therapies of Organic Fluids by Using Extracorporeal Circuits

(71) Applicants: Corrado Bellini, Medolla (IT); Daniele Galavotti, Mirandola (IT); Claudio Ronco, Vicenza (IT)

(72) Inventors: Corrado Bellini, Medolla (IT); Daniele Galavotti, Mirandola (IT); Claudio Ronco, Vicenza (IT)

(73) Assignee: RAND S.R.L., Medolla (IT)

(21) Appl. No.: 13/876,087

(22) PCT Filed: Sep. 28, 2011
(86) PCT No.: PCT/IB11/54261

§ 371 Date: Apr. 17, 2013

(30) Foreign Application Priority Data
Sep. 29, 2010 (IT) MO2010A000270

(51) Int. Cl. A61M 1/36

(52) U.S. Cl. CPC A61M 1/3621
USPC 210/96.2; 210/416.1

(57) ABSTRACT

A wearable system for performing purifying therapies on organic fluids with the use of extracorporeal circuits includes a clothing article wearable by a patient, in which at least one disposable extracorporeal circuit is removably housed. The wearable clothing article has a line for drawing, from a patient drawing point, organic fluid to be purified, and a line for returning, to a patient infusion point, purified organic fluid. A filter is included for filtering the organic fluid to be purified, arranged between the drawing line and the return line. A first pump is provided for pumping, along the extracorporeal circuits, the organic fluid to be purified and the purified organic fluid. A means for collecting and storing fluid residues filtered from the organic fluid to be purified, is connected to the filter via a collection line. The extracorporeal circuit includes an anti-thrombogenic covering.

### FIELD OF APPLICATION

The invention regards a wearable system for executing depurative treatments of organic fluids with the use of extracorporeal circuits, in particular for executing hemofiltration therapies allowing patients to walk around during treatment and be independent of fixed machines and hospitals; in this manner, the quality of life of the patients is improved.

### STATE OF THE ART

Conventionally, all the treatments that use extracorporeal circuits, such as hemofiltration therapies of the blood, but also other temporary treatments of depurative type, are executed with bulky equipment pieces in which various devices are comprised; such devices are similar to each other and are also present on the market as devices that have their own independent functioning.

As said, these equipment pieces use extracorporeal circuits in which the various components are connected to each other without a specific structural integration, or in any case with a very limited integration. This circumstance occurs in order to give users the opportunity to mount, on one same equipment piece, disposable devices with equivalent performances but that are made by different producers.

Normally, an extracorporeal circuit comprises a double-lumen venous catheter that is percutaneously applied on the patient on a vascular access of average size, e.g. the jugular vein.

These catheters are typically elements that allow the disposable circuit mounted on the equipment piece and the patient to be interfaced with each other.

Overall, the circuit consists of a pump, which by means of a suitable duct channels the blood arriving from the patient towards a hemofilter.

The circuit also comprises a pump, which extracts an aqueous fraction of the blood from the blood moving through the filter, sending such fraction to a separated aqueous fraction collection system.

Finally, there is a further duct for returning the filtered blood to the patient, as well as, typically, a series of further ducts that have the task of interconnecting all these elements together.

In these known circuits, the possibility of employing a wide variety of devices on a single equipment piece, in addition determining, as said, a limited possibility of integration among the various disposable devices, also determines a greater possibility of error induction by the operators assigned to use the equipment pieces. Hence, there is a serious risk of compromising the safety of the patients subjected to the treatments.

It must be briefly recalled that the therapeutic grounds of a hemofiltration treatment derive from the need to remove an excess of liquids in the patients; such liquid excess can cause considerable metabolic disorders, renal deficit, and especially a congestive cardiac insufficiency.

Due to a limited capacity for treatment with ACE inhibitor drugs, with diuretic drugs and with beta blockers, which do not totally solve the problem, the depurative techniques that employ convective removal of fluids from the blood, such as ultrafiltration, have been used for improving the capacity to combat this pathology type.

According to the patent application US2008/0051689 in the name of Gura, ari ultrafiltration device is known for extracting a predetermined fluid volume from the blood of patients to be treated, using a precise control system.

The device comprises an adaptable belt, capable of being worn by the patients (with weight less than 2 pounds, i.e. about 0.9 kilograms) that is equipped with fixing means between the two ends.

Inside the area delimited by the belt, there is a hemofilter which is connected to a vascular access of the patient and a pump which drives the blood drawn from the patient towards the hemofilter and which reinfuses the blood in the patient by means of a suitable duct, through a second vascular access.

Between the pump and the hemofilter, there is a further micropump for metering an anticoagulant substance, functional for inhibiting coagulation and platelet aggregation, which are natural effects characterizing the phenomenon of contact between blood and artificial or extracorporeal surfaces in general; hence such substance maintains open, i.e. free, the entire hydraulic circuit crossed by the blood.

The pump and the micropump are power-supplied by a group of batteries, also mounted on the belt.

The hemofilter has the task of eliminating the water load excess connected to the above-described pathologies, by separating the aqueous fraction from the blood of the patients.

In a simpler embodiment of the belt, this liquid is collected due to gravity in a suitable collection bag that does not have to be connected to the belt; for example, it could be connected to one of the legs of the patient, in a manner so as to utilize the pressure gradient that facilitates the collection of the aqueous fraction filtration component.

In two further possible embodiments according to the Gura patent application, the hemofilter is primarily coupled with other analogous devices, which are coupled in series or in parallel with each other, in which the aqueous fraction filtered from the various filters is in any case collected in a single reservoir.

According to a more developed version, a control unit is also provided for, it too power-supplied by the same batteries used by the blood pump.

The battery group can be recharged separately from the belt, or while it is mounted on the latter during use, and the autonomy of the batteries is such to allow the execution of at least 6 hours of continuous treatment of the entire ultrafiltration device.

The presence of a humidity sensor is provided for, which is connected with a control unit and which is housed in the structure that encloses the entire device. Such sensor has the task of detecting the appearance of moisture or condensate, which are an indirect signal of the occurrence of possible leaks of blood or filtered liquid from the system.

The control unit manages the blood pump by regulating its pumping power, volume and frequency over the unit of time, and thus actually regulating the pressure of the blood circulating in the device.

The flow of the pump can therefore be automatically adjusted by the control unit, and also manually adjusted by operating on a control, normally a potentiometer, which is activatable by the user or by the doctor responsible for the therapy.

A further element characterizing the device is the presence of a suitable pump for pumping an anticoagulant drug into the circuit and the relative container for storing and controlling the volume of the stored drug, by means of a level sensor contained in the anticoagulant container.

The anticoagulant pump meters the anticoagulant drug during the passage of the blood through the circuit, introducing it between the blood pump and the inlet of the hemofilter.

The aqueous fraction filtered from the hemofilter is collected in a bag and/or a reservoir which can also be arranged in a specific position with respect to the position of the hemofilter, in order to utilize the hydraulic head thereof.

Inside the bag and/or reservoir, a level sensor is housed that is connected to the control unit, in a manner so as to send an alarm signal to the users upon attaining a pre-established volume; if required, the system can be completely stopped if this alarm is not reset within a pre-established time interval.

The bag/reservoir can contain an absorbent material at its interior, such as foam or a sponge, in order to limit the fluctuation of the movement of the liquid volume that is determined according to the mobility and relative position of the user with respect to the bag/reservoir.

The latter is equipped with filtered volume evacuation members which can be activated by an electromechanical valve commanded by the control unit, or more simply the bag/reservoir can be completely removable or disposable in order to be replaced with an equivalent empty bag/reservoir.

According to one variant of the Gura patent application, there is a small collection bag at the filtered liquid outlet, connected to a suction micropump which has the function of improving and regulating the filtering capacity of the filter, in addition to pumping the sucked liquid into a bag coupled or housed inside the belt. Also in this case, the bag/reservoir can contain an absorbent material therein, such as a sponge or foam, necessary for limiting the fluctuation of the movement of the liquid volume that is determined according to the mobility and relative position of the user with respect to such bag, and a level sensor or a pressure sensor capable of communicating the attainment of a specific volume to the control unit.

This state of the art has several drawbacks.

A first drawback consists of the fact that the known device according to the Gura patent application is not equipped with a monitoring of the pressures that can develop in the circuit, and this renders the hemofiltration therapy considerable less precise.

Another drawback consists of the fact that there is no sensor for detecting possible blood losses on the filtered liquid passage line, and therefore losses cannot be detected, hemofilter fiber breakage cannot be prevented and the occurrence of hemolytic events caused by device functioning problems cannot be monitored.

A further drawback is the lack of a sensor for detecting air microbubbles on the filtered blood reinfusion line to the patient.

Another drawback is the lack of a safety closure device for the reinfusion line to the patient, which is driven by the control unit in case of detection of microbubbles by the detection sensor.

A further drawback is that an anticoagulant drug must be used, and this requires the presence of an additional wearable element, in addition to the normal devices for executing the hemofiltration, which consists of an anticoagulant pump and the relative members for storing the drug and controlling the volume stored in the reservoir, as well as the members provided for metering the anticoagulant during the treatments; all of the above renders the device more complex, bulky and heavy.

### PRESENTATION OF THE INVENTION

One object of the present invention is to improve the state of the art. Another object of the invention is to obtain a wearable system for executing depurative treatments of organic fluids with the use of extracorporeal circuits which allows considerably simplifying the structure of the extracorporeal circuit, in a manner such to be able to integrate it into a clothing article that can be easily worn and borne by patients undergoing therapies that require the use of extracorporeal circuits, so as to render the patients completely independent of fixed machines and hospitals/clinics.

Still another object of the invention is to obtain a wearable system for executing depurative treatments of organic fluids with the use of extracorporeal circuits that allows both patients and medical personnel to constantly keep the therapy procedure under control, even at a distance.

A further object of the invention is to obtain a wearable system for executing depurative treatments of organic fluids with the use of extracorporeal circuits that has maximum safety of use for patients, preventing the risks of reinfusion of treated blood containing air microbubbles.

Another object of the invention is to obtain a wearable system for executing depurative treatments of organic fluids with the use of extracorporeal circuits that allows maintaining the pressure values inside the extracorporeal circuits under control at any moment.

A further object of the invention is to obtain a wearable system for executing depurative treatments of organic fluids with the use of extracorporeal circuits that does not require the use of anticoagulant drugs and devices for administering the latter.

According to one aspect of the invention, a wearable system is provided for executing depurative treatments of organic fluids with the use of extracorporeal circuits, in accordance with claim 1.

The invention therefore allows attaining the following advantages:
obtaining a wearable system for executing depurative treatments of organic fluids with the use of extracorporeal circuits that allows considerably simplifying the structure of the extracorporeal circuit, in a manner so as to be able to integrate it into a clothing article that can be easily worn and borne by patients subjected to therapies that require the use of extracorporeal circuits, so as to render the patients completely independent from fixed machines and hospitals/clinics;
obtaining a wearable system for executing depurative treatments of organic fluids with the use of extracorporeal circuits that allows both patients and medical personnel to constantly keep the therapy status under control, even at a distance;
obtaining a wearable system for executing depurative treatments of organic fluids with the use of extracorporeal circuits that that has maximum safety of use for patients, preventing the risks of reinfusion of treated blood containing air microbubbles;
obtaining a wearable system for executing depurative treatments of organic fluids with the use of extracorporeal circuits that allows maintaining the pressure values inside the extracorporeal circuits under control at any moment;
obtaining a wearable system for executing depurative treatments of organic fluids with the use of extracorporeal circuits that does not require the use of anticoagulant drugs and devices for administering the latter, rendering the wearable system more compact and lighter.

### BRIEF DESCRIPTION OF THE DRAWINGS

Further characteristics and advantages of the invention will be clearer from the detailed description of a preferred but not exclusive embodiment of a wearable system for executing depurative treatments of organic fluids with the use of extracorporeal circuits, illustrated as a non-limiting example in the drawing table set, in which:

FIG. 1 is a schematic front view of a possible embodiment of a wearable system, specifically a vest, for executing depurative treatments of organic fluids with the use of extracorporeal circuits, according to the invention;

FIG. 2 is a rear view of the system of FIG. 1;

FIG. 3 is a schematic view of an extracorporeal circuit for executing depurative treatments of organic fluids, which is housable inside the vest of FIG. 1.

### DETAILED DESCRIPTION OF A PREFERRED EMBODIMENT

With reference to the figures, a clothing article is indicated with 1, in the specific case a vest, which can be worn by a person, specifically by a patient “P” who must undergo an organic fluid depuration therapy, for example blood depuration, known as hemofiltration.

The vest 1 comprises a body piece 2 in which a series of pockets are made, both on the front side and on the rear side, whose function will be better explained hereinbelow.

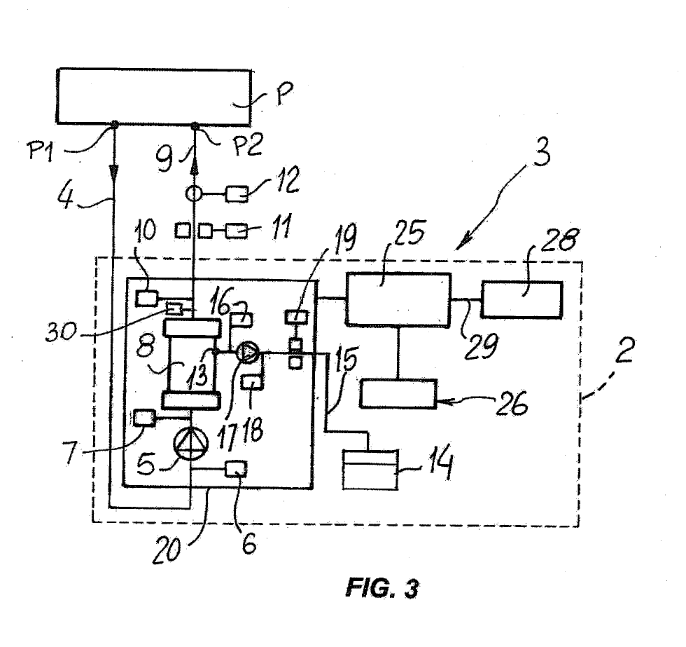

With reference to FIG. 3, it is noted that an extracorporeal circuit for executing a depuration treatment of an organic fluid, for example blood (not the only possibility), is indicate with 3 and comprises a drawing line 4 for drawing the blood to be depurated from a drawing point “P1” of the patient “P”.

The drawing line 4 is coupled onto the body of a pump 5, which is configured to make the blood circulate in the extracorporeal circuit 3, crossing a drawing pressure sensor 6.

From the pump 5, the line 4 crosses a pre-filtration pressure sensor 7, and downstream of the latter, is connected with a hemofilter 8, in which the filtration of the blood occurs as well as the separation of the excess aqueous components.

The hemofilter 8 has an outlet port from which a return line 9 departs to the patient “P”, in order to reinfuse the depurated blood in the latter through a second access point “P2”.

At the outlet of the hemofilter 8, a return or reinfusion pressure sensor 10 is mounted on the return line 9; downstream of such sensor 10, a detection sensor 11 is mounted, configured to detect the presence of air in the venous blood that returns to the patient “P”, as is a clamp safety element 12 that is intended to close the return line 9 if the detection sensor 11 detects the presence of air in the blood or in the return line 9.

The hemofilter 8 also has a discharge port 13 through which the liquid filtered from the blood is discharged into one or more collection bags 14 via a discharge line 15 that departs from the discharge port 13 and terminates in the collection bag 14.

On this discharge line 15, downstream of the discharge port 13, a filtered liquid pressure sensor 16 is mounted; downstream of the latter, a second pump 17 is mounted, configured to suck the filtered liquid from the hemofilter 8 and discharge it into the collection bag 14.

Downstream of the second pump 17, on the discharge line 15, a detection sensor 18 is mounted for detecting the pressure of the filtered liquid being discharged and, between the latter and the collection bag 14, a loss detector 19 is mounted, in the specific case a blood loss detector.

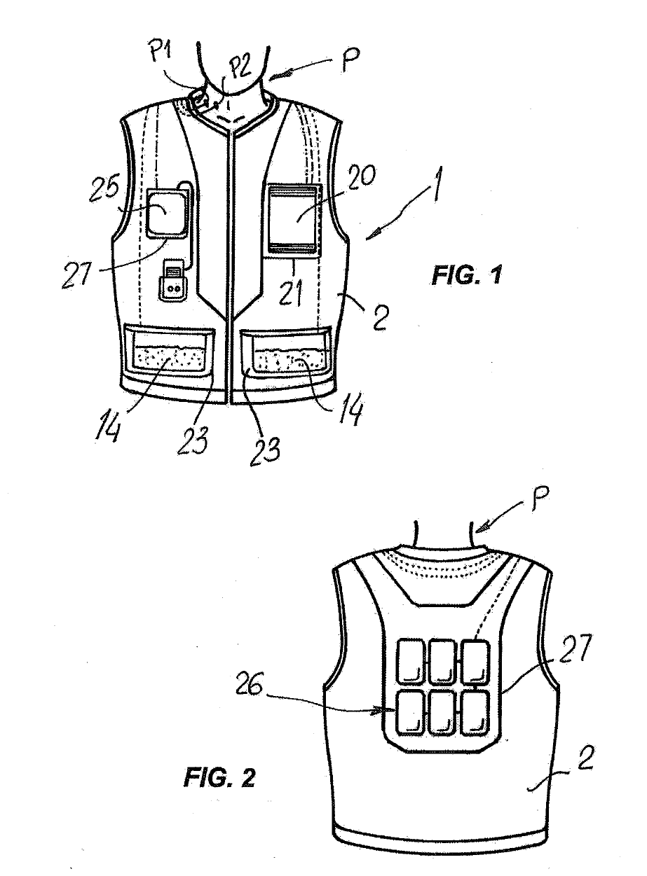

It must be noted that, in accordance with the invention, the hemofilter 8, the pump 5, the drawing pressure sensor 6, the pre-filtration pressure sensor 7, the reinfusion pressure sensor 10, the two pressure sensors placed on the filtered liquid line, 16 and 18, one placed before the pump 17 and the second placed after the aforesaid pump, and the blood loss detector 19 are joined in a single common body, which is schematically indicated in FIG. 3 with the rectangle 20. Such common body has flattened shape, in a manner such that it can be contained in one of the pockets of the body piece 2; in the specific case, with reference to FIG. 1, it is contained in the front pocket 21.

The collection bag 14 can be a double bag and each of the bags 14 can be contained inside respective front pockets 22 and 23 of the body piece 2.

The ducts which form the extracorporeal circuit 3 are inserted in the body piece 2, for example in an interspace defined between the lining of the body and its external fabric.

With reference once again to FIG. 3, it is observed that the extracorporeal circuit 3 also provides for a general electronic control unit 25, indicated in brief below as control unit 25; such unit is connected to the above-indicated elements that are joined together in the common single body 20, and it controls and commands the functioning of the pump 5 and the second pump 17 and also acquires the signals coming from the pressure sensors and that of blood presence from the blood loss detector 19.

The control unit 25 is contained inside a pocket 28 provided on the front side of the body piece 2.

The latter pumps and the control unit 25 are power-supplied with a group of batteries 26, normally of rechargeable type, which can be arranged in a suitable pocket 27 obtained on the rear of the body piece 2, as indicated in FIG. 2.

The extracorporeal circuit 3 also comprises a drive unit 28 which is separate from the control unit 25 but connected thereto via a connection line 29, and which can be directly handled by the patient “P” in order to modify, if required, the functioning parameters of the pumps 5 and 17.

The drive unit 28 can also display parameters relative to the execution of the depuration therapies, such as the pressure values along the extracorporeal circuits, the treatment time, the charge state of the batteries 26 and alarms determined by irregular conditions.

According to a more complete version, not illustrated, the extracorporeal circuit 3 can be equipped with a transceiving device connected with the control unit 25 for the remote transmission to medical operators of the parameters detected during the execution of the organic fluid depurative therapies.

In addition, normally on the return line 9, a chemical parameter detection point can also be provided, indicated with 30 in the FIG. 3, which in the case of blood depuration can supply hematochemical parameter values.

The functioning of the wearable system for executing depurative treatments of organic fluids with the use of extracorporeal circuits is indicated below for executing, as an example, hemofiltration therapies. Such functioning is as follows: the blood to be depurated of an excess water quantity is drawn from the drawing point “P1” of a patient “P” and through the drawing line 4 is introduced into the pump 5, previously passing through the pressure sensor 6 which detects the pressure inside the line 4 and transmits the value thereof to the general electronic control unit 25.

The pump 5 drives the blood to be depurated towards the hemofilter 8 and the pre-filtration sensor 7 in turn detects the pressure inside the line 4, in the segment between the pump 5 and the inlet of the hemofilter 8, once again transmitting the detected pressure data to the control unit 25.

Inside the hemofilter 8, the moving blood is depurated of the excess water and is re-sent to the patient “P” via the return line 9 that flows into the access point “P2”.

Passing along the return line 9, the depurated blood is checked by the detection sensor 11, which controls that there is no trace of air therein before the depurated blood reaches the access point “P2”; a signal is sent to the control unit 25.

If the sent signal confirms that there is no trace of air therein, the blood flow inside the return line 9 is maintained, while in the opposite case, the control unit 25 immediately activates the clamp safety element 12 that completely closes the return line 9 in order to prevent the air from being infused into the circulatory apparatus of the patient “P”, creating a serious risk for the patient's safety.

The filtered liquid is expelled by the hemofilter 8 through the discharge line 15 which is connected to the latter via the discharge port 13.

The expulsion occurs via the action of the second pump 17, which sucks the filtered liquid and sends it towards the collection bags 14.

The filtered liquid pressure sensor 16 detects the pressure thereof upstream of the second pump 17 and sends the detected parameter to the control unit 25.

Downstream of the second pump 17, the pressure sensor of the filtered liquid being discharged detects the pressure value thereof and sends the data to the control unit 25.

Finally, before reaching the bags 14, the filtered liquid passes through the blood loss detector 19, which also sends a signal to the control unit 25.

If the signal confirms that there are no blood losses or hemolytic phenomena underway at the hemofilter, the control unit maintains the second pump 17 in function, whereas it blocks the functioning thereof if such losses/phenomena are confirmed.

All the signals and data collected by the control unit 25 can be visually controlled by the patient “P” and by the medical operators during the course of the hemofiltration therapy; when the mounting of a transceiving device is provided, the data can also be read remotely, without the patient “P” having to remain in a hospital.

The patient “P” can control the functioning of the pumps 5 and 17, modifying their pumping rate by operating on the drive unit 28.

It must be underlined that the execution of the hemofiltration therapy is particularly easy and non-traumatic for the patient, who after having put on the body piece 2, can carry out his own daily activities without having to remain inside a hospital.

The grouping in the common body 20 of most of the devices that are necessary for executing the hemofiltration therapies, or generally depurative devices, allows rendering the body piece 2 particularly comfortable and light to wear.

In addition, the anti-thrombogenic covering of the entire surface of the extracorporeal circuit in contact with the organic fluid is obtained by chemically immobilizing molecules of heparin or biomimetic substances that render the surfaces inert. This allows eliminating from the extracorporeal circuit 3 the anticoagulant substance storage tank and the additional pump for the infusion of the anticoagulant substance into the extracorporeal circuit 3.

The man skilled in the art can comprehend that the vest 1 can also be substituted by another garment, such as a conventional jacket for a normal suit or for normal clothes, or a windbreaker, an overcoat and the like.

It must also be noted that the single body 20 and the bags 14 are of disposable type and can be extracted from the respective containment pockets 21, 22, 23, disconnected from the control unit 25 and removed at the end of each therapy cycle.

The man skilled in the art can comprehend that a disposable extracorporeal circuit can also comprise, if required, one or more drawing points for sampling the blood and the filtered liquid, in order to monitor the hematochemical parameters by means of integrated sensors and biochips.

It has been established, in practice, that the invention attains the pre-established goals.

The invention as conceived is susceptible to modifications and variants, all falling within the inventive concept.

In addition, all the details can be substituted with other technically equivalent elements.

In practice, the materials employed as well as the shapes and size can be of any type, according to requirements, without departing from the protective scope of the following claims.

1. A wearable system for performing purifying therapies of organic fluids by using extracorporeal circuits comprising:
An article of clothing wearable by a patient, wherein at least a disposable extracorporeal circuit can be arranged in a removable way, said disposable extracorporeal circuit comprising:
A drawing-line of organic fluid to be purified from a drawing point of a patient;
A return-line of purified organic fluid to an infusion point of the patient;
Filter means to filter said organic fluid to be purified, arranged between said drawing-line and said return-line;
First pumping means to pump both said to be purified and purified organic fluid along said extracorporeal circuits;
Collecting and amassing means of fluid residues filtered from said organic fluid to be purified, connected to said filter means by a collecting-line;
wherein said extracorporeal circuit comprises an anti-trombogenic coating.

2. A wearable system according to claim 1, wherein said anti-trombogenic coating is applied on every surface of said extracorporeal circuit in contact with said organic fluid to be purified and purified.

3. A wearable system according to claim 1, wherein it further comprises control means of the working of at least said first pumping means.

4. A wearable system according to claim 1, wherein first pressure detecting means of said organic fluid to be purified are mounted on said drawing-line.

5. A wearable system according to claim 1, said first pressure detecting means are placed upstream said first pumping means.

6. A wearable system according to claim 1, wherein second pressure detecting means of said organic fluid to be purified are mounted on said drawing-line, between said first pumping means and said filter means.

7. A wearable system according to claim 1, wherein third pressure detecting means of said purified organic fluid are mounted on said return-line.

8. A wearable system according to claim 1, wherein air detecting means in said purified organic fluid are mounted on said return-line.

9. A wearable system according to claim 8, wherein blocking means of flowing of said purified organic fluid which can be actuated by said air detecting means in an air detection condition are mounted on said return-line.

10. A wearable system according to claim 1, wherein chemical parameters detecting means are mounted on said return-line.

11. A wearable system according to claim 1, wherein pressure detecting means of said fluid residues are mounted on said collecting-line.

12. A wearable system according to claim 1, wherein leakage detecting means of said organic fluid and/or said fluid residues are mounted on said collecting-line.

13. A wearable system according to claim 1, wherein second pumping means are mounted on said collecting-line so configured to pump said fluid residues into said collecting and amassing means.

14. A wearable system according to claim 3, wherein said control means comprise a remote control unit which can be operated by the patient and/or health operators.

15. A wearable system according to claim 1, wherein said fluid to be purified is blood.

16. A wearable system according to claim 1, wherein said filter means comprise a hemo-filter.

17. A filter apparatus mountable on extracorporeal circuits for purification of organic fluids, further comprising filtering means housing body which can be crossed by said organic fluids to be purified, said housing body comprising, arranged in an integrated configuration:
A first pump for pumping said organic fluids to be purified along said extracorporeal circuits from a drawing point of a patient to a re-infusion point of purified organic fluids of said patient;
A second pump for pumping away from said filtering means filtered substances from said organic fluids;
A plurality of chemical parameters detecting means to be fitted along said extracorporeal circuits;
Leakage detecting means of said organic fluids to be purified/purified.

18. A filter apparatus according to claim, wherein said plurality of chemical parameter detecting means is selectively connectable with said first pump and second pump, so as to control their working.
